(19) United States
(12) Patent Application Publication
(10) Pub. No.: US 2016/0160211 A1
(43) Pub. Date: Jun. 9, 2016

(54) TWO DIMENSIONAL CAPILLARY ELECTROPHORESIS APPARATUS

(71) Applicant: UNIVERSITY OF NOTRE DAME DU LAC, Notre Dame, IN (US)

(72) Inventors: Bonnie Jaskowski Huge, South Bend, IN (US); Ryan John Flaherty, South Bend, IN (US); Oluwatosin O. Dada, Everett, WA (US); Norman J. Dovichi, South Bend, IN (US)

(73) Assignee: UNIVERSITY OF NOTRE DAME DU LAC, Notre Dame, IN (US)

(21) Appl. No.: 14/873,319

(22) Filed: Oct. 2, 2015

Related U.S. Application Data
(60) Provisional application No. 62/058,715, filed on Oct. 2, 2014.

(51) Int. Cl. C12N 15/10; G01N 27/447

(52) U.S. Cl. CPC C12N 15/1048; G01N 27/44791; G01N 27/44773

(57) ABSTRACT

The present disclosure describes an apparatus for identifying a polynucleotide capable of binding a target. The apparatus comprises a first, second, and third high voltage power supply; a first, second, and third capillary tube; a first, second, and third buffer reservoir; a fraction collector, and at least one collection vessel.

### CROSS-REFERENCE TO RELATED APPLICATIONS

This application claims the benefit of and priority to U.S. Provisional Application Ser. No. 62/058,715 filed on Oct. 2, 2014.

### STATEMENT REGARDING FEDERALLY SPONSORED RESEARCH OR DEVELOPMENT

This invention was made with government support under 1R21RR032362-01 awarded by the National Institutes of Health. The government has certain rights in the invention.

### REFERENCE TO A SEQUENCE LISTING

The Sequence Listing, which is a part of the present disclosure, includes a computer readable form comprising nucleotide and/or amino acid sequences of the present disclosure. The subject matter of the Sequence Listing is incorporated herein by reference in its entirety.

### BACKGROUND

1. Field

The present disclosure relates to an apparatus that couples three capillary tubes together in a substantially co-axial fashion and utilizes a fraction collector to collect components exiting the third capillary tube. The present disclosure further relates to the method of operating the disclosed apparatus.

2. Description of Related Art

Coupling two capillary tubes together for various purposes has been disclosed previously. One example of such a disclosure is U.S. Pat. No. 5,798,032. Reasons for coupling two capillary tubes together include allowing the transfer of a selected component from a first capillary tube to a second capillary tube, or to introduce a supplementary reagent, such as an internal standard, binding agent, or enzyme into the system.

The coupling of two capillary tubes together can be accomplished in several ways. Examples of ways to couple two capillary tubes together are disclosed in U.S. Pat. No. 4,936,974. One method for coupling two capillary tubes together includes inserting a portion of a first capillary tube into a portion of a second capillary tube to define an overlapping section that results in a coupling of the two capillary tubes. Another method for coupling two capillary tubes together includes placing two capillary tubes of similar inner diameters end-to-end such that opposing ends of the capillaries are adjacent rather than overlapping. Yet another method for coupling two capillary tubes together includes forming an aperture in the wall of a single capillary tube such that the single capillary tube can be treated as two capillary tubes.

In each of the coupling arrangements of the foregoing disclosures, a fluid can be introduced at the junction between the two capillary tubes and the fluid can comprise a supplementary reagent. Additionally, in the foregoing disclosures a first capillary tube can be used to separate components of a mixture and a second capillary tube can be used to mix a supplementary reagent with a sample component or to detect a sample component that has been labeled in some fashion by the supplementary reagent. If the supplementary reagent mixed with the sample component is a binding agent, then there still exists a need for the further separation of the sample mixture so as to distinguish between bound and unbound components. Additionally, the collection of the separated component that is either bound or unbound to the binding agent is desirable for further characterization or manipulation.

### SUMMARY

The present disclosure provides, in one embodiment, an apparatus for identifying a polynucleotide capable of binding a target. The apparatus can comprise a first high voltage power supply connected to a first high voltage electrode. The first high voltage electrode can be inserted into an injection block comprising a first buffer reservoir. The apparatus can further comprise a first capillary tube having a first end of said first capillary tube inserted into said injection block and submerged in said first buffer reservoir. The apparatus can further comprise a second high voltage power supply connected to a second high voltage electrode submerged in a second buffer reservoir. The apparatus can further comprise a third high voltage power supply connected to a third high voltage electrode submerged in a third buffer reservoir. The apparatus can further comprise a first buffer channel and a first waste outlet. The apparatus can further comprise a second capillary tube having a substantially co-axial alignment with said first capillary tube. The apparatus can further comprise a first four-way junction between a second end of said first capillary tube, a first end of said second capillary tube, said first buffer channel, and said first waste outlet. The first four-way junction can be connected to said second buffer reservoir via said first buffer channel. The apparatus can further comprise a second buffer channel and a second waste outlet. The apparatus can further comprise a second four-way junction between a second end of said second capillary tube, a first end of a third capillary tube, said second buffer channel, and said second waste outlet. The second four-way junction can be connected to said third buffer reservoir via said second buffer channel. The injection block can be fluidly attached to an injector capable of injecting fluid into said first end of said first capillary tube proximate to said injection block.

In the apparatus, the first buffer channel and said first waste outlet can be perpendicular to said first capillary tube and said second capillary tube. In the apparatus, said second buffer channel and said second waste outlet can be perpendicular to said second capillary tube and said third capillary tube. In the apparatus, said second buffer channel and said second waste outlet can be parallel to said first buffer channel and said first waste outlet. The apparatus can further comprise a fraction collector fluidly connected to a distal end of the third capillary tube proximate to the fraction collector. The apparatus can further comprise a computer program capable of controlling said first high voltage power supply, said second high voltage power supply, said third high voltage power supply, said injector, and said fraction collector. In the apparatus, at least one of the first and second four-way junctions can be sealed.

The present disclosure provides, in an additional embodiment, an apparatus for identifying a polynucleotide capable of binding a target. The apparatus can comprise a first four-way junction, a second four-way junction, a first capillary tube, a second capillary tube, and a third capillary tube. The first, second and third capillary tubes can each have a first and a second end. The first capillary tube, said second capillary tube, and said third capillary tube can be aligned in a substantially co-axial alignment to one another. The second end of said first capillary tube and said first end of said second capillary tube can be fluidly attached at said first four-way junction; and said second end of said second capillary tube and said first end of said third capillary tube can be fluidly attached at said second four-way junction. The second end of said third capillary tube can be connected to a fraction collector by a first port of a “T” fitting. The apparatus can further comprise a nozzle fluidly attached to a second port of said “T” fitting. The nozzle can be connected to an electrical ground by a grounding wire. The fraction collector can be supplied with a sheath liquid. The sheath liquid can be filtered by an inline filter fluidly connected to a sheath buffer reservoir. The inline filter can be fluidly connected to a computer-controlled valve that is itself fluidly connected to a third port of said “T” fitting. The apparatus can further comprise a collection vessel on a translational stage. The translational stage can be configured to move in at least one of the X, Y, and Z directions, and be disposed to receive fluid dispensed from said nozzle.

In the apparatus, the first buffer channel and said first waste outlet can be perpendicular to said first capillary tube and said second capillary tube. In the apparatus, the second buffer channel and said second waste outlet can be perpendicular to said second capillary tube and said third capillary tube. In the apparatus, the second buffer channel and said second waste outlet can be parallel to said first buffer channel and said first waste outlet. The apparatus can further comprise a fraction collector fluidly connected to a second end of said third capillary tube. The apparatus can further comprise a computer program capable of controlling said first high voltage power supply, said second high voltage power supply, said third high voltage power supply, said injector, and said fraction collector. In the apparatus, the collection vessel can be a 96-well plate. In the apparatus, at least one of the first and the second four-way junction can be sealed. In the apparatus, the sheath buffer reservoir can be held under pressure greater than atmospheric pressure, and the pressure can be pressurized by nitrogen gas.

### BRIEF DESCRIPTION OF THE DRAWINGS

FIG. 1 is a schematic view of a capillary electrophoresis apparatus in accordance with the present disclosure.

FIG. 2 shows a flowchart for the operation of one embodiment of the present disclosure.

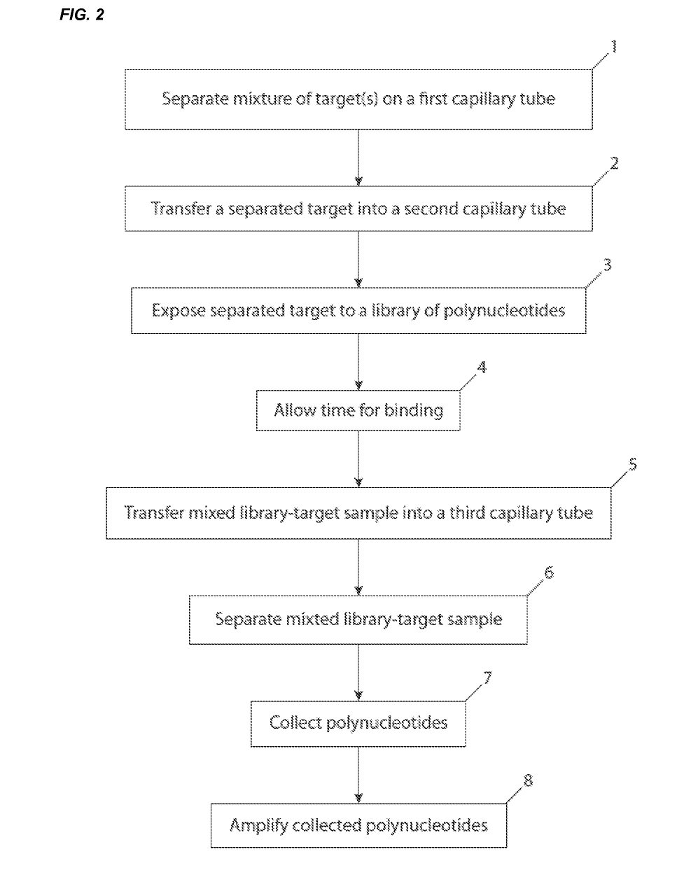

FIG. 3 is an electropherogram of a fluorescently labeled polynucleotide after being subjected to the operation of the apparatus of the present disclosure.

FIG. 4 is an electropherogram of a fluorescently labeled polynucleotide in the presence of the target to which the polynucleotide corresponds after being subjected to the operation of the apparatus of the present disclosure.

FIG. 5 is a schematic view of a capillary electrophoresis-coupled fraction collector in accordance with the present disclosure.

FIG. 6 is a schematic view of a capillary electrophoresis apparatus comprising a single separation capillary tube and a fraction collector in accordance with the present disclosure.

### DETAILED DESCRIPTION

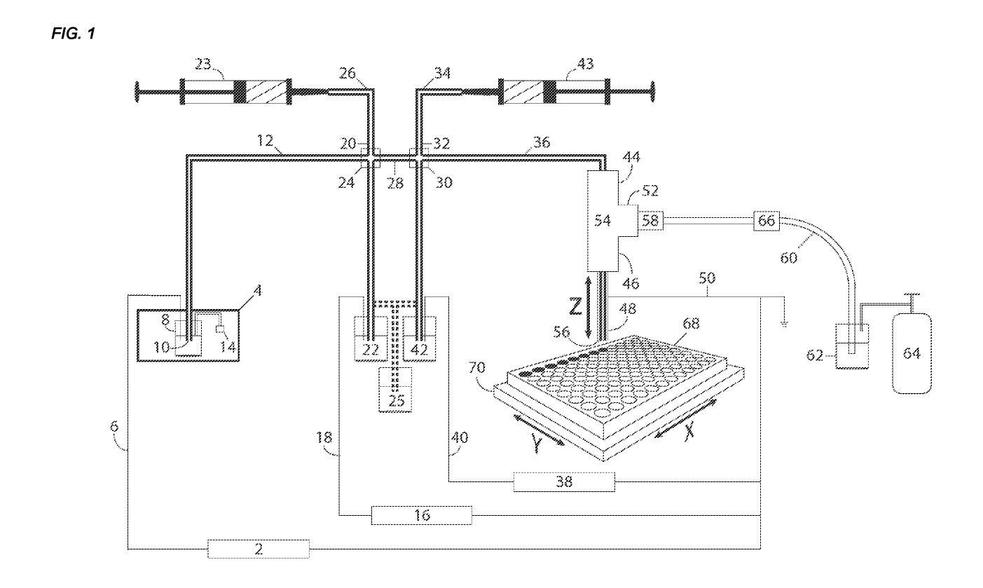

FIG. 1 shows an embodiment of the disclosed Two-dimensional Capillary Electrophoresis Systematic Evolution of Ligands by Exponential Enrichment (2D-CE-SELEX) apparatus. A first high voltage power supply (2) is connected to an injection block (4) by a first high voltage electrode (6) that is electrically conductive. The injection block (4) contains a first buffer reservoir (8). The injection block (4) brings together, in a subassembly of the 2D-CE-SELEX apparatus, the first buffer reservoir (8), the first high voltage electrode (6), a first end of the first capillary tube (10), and an injector (14) capable of injecting fluid into the first end of the first capillary tube (10). The fluid injected into the first end of the first capillary tube (10) can be an electrically conductive liquid. The injection block (4) generally can be an assembly that facilitates: the introduction of a fluid, typically sample or buffer, into the first end of the first capillary tube (10) by the injector (14), holds the first high voltage electrode (6) at a depth in the first buffer reservoir (8) sufficient to establish an electrical connection between the first high voltage power supply (2) and the first buffer reservoir (8), and holds the first end of the first capillary tube (10) at a depth in the first buffer reservoir (8) sufficient to establish an electrical connection between the first high voltage power supply (2) and a first capillary tube (12).

The term “electrically conductive” is intended to define a material capable of transmitting a voltage applied by a power supply to an environment, e.g. a buffer reservoir, which is external to the power supply. Electrically conductive materials include, but are not limited to, copper, aluminum, platinum, stainless steel, and the like. Electrical conductivity can also be established by materials that are not solid at room temperature, for example, by liquids. Electrical conductivity can be formed between two environments when the environments are fluidly connected by an electrically conductive medium. For example, a capillary tube filled with a liquid that contains ions can be electrically conductive and establish an electrical connection between two remote environments, e.g., between a first buffer reservoir and a second buffer reservoir.

The buffer solutions used in the buffer reservoirs can be aqueous solutions containing ions. Buffer solutions generally have the characteristic of resisting changes in pH, which is known as buffering capacity. Buffer solutions often consist of a mixture of weak acid and its conjugate base, or vice versa. Buffering capacity of buffer solutions results from the equilibrium that is present between the acid and its conjugate base. The acid and conjugate base often have counter ions to balance the charge in solution and are therefore electrically conductive as a result of the ions present in the solution. Buffer solutions need not be entirely aqueous solutions and can include non-aqueous components including, but not limited to, acetonitrile, methanol, acetone, and the like.

The first buffer reservoir (8), the second buffer reservoir (22), and a third buffer reservoir (42) can be any non-conductive material impervious to a liquid, including but not limited to polymer-based materials, plastic, and glass. In one embodiment, the buffer reservoirs can be plastic, specifically, polypropylene. Other materials can be used and one of skill in the art will appreciate that a major consideration in determining the material used to make the buffer reservoirs is that the material be chemically inert to the buffer being placed in the reservoir. A sheath liquid reservoir (62) can be made of glass, sealed, and held under pressure from a compressed gas cylinder (64). The first buffer reservoir (8) can be sealed upon attachment of the bottom portion of the injection block to the rest of the injection block (4). The second buffer reservoir (22) and the third buffer reservoir (42) can remain open to atmospheric pressure. One of skill in the art will recognize that the choice of whether to seal or leave a reservoir open to atmospheric pressure will be determined by the design of the apparatus and the purpose that the reservoir serves.

The first high voltage electrode (6) and the first end of the first capillary tube (10) are submerged in the first buffer reservoir (8) to a depth that is sufficient to establish an electrical connection between the first high voltage power supply (2) and the first capillary tube (12). When a voltage is applied to a length of capillary tubing an electric field is established, which is the voltage drop across the length of capillary tubing. The electric field affects the mobility of the fluid present in the length of capillary tubing. Capillary tubes frequently have an inner diameter between 1 μm and 200 μm; however inner diameters outside of this range are available and can be used with the disclosed apparatus. Altering the disclosed apparatus to use different sizes of capillary tubes is readily appreciated by one of skill in the art. The inner diameter of the capillary tube is preferably less than or equal to 100 μm when the apparatus is used for the separation of analytes within the capillary tube. When the inner diameter of the capillary tube is less than or equal to 100 μm the electro-osmotic flow acts uniformly throughout the capillary tube cross section and prevents convection-induced zone broadening. The outer diameter of the capillary tube varies and should be chosen such that the capillary tube can be manipulated without breaking. Overall, the capillary tube should have a high external-surface-area-to-internal-volume ratio to facilitate efficient heat transfer. Such efficient heat transfer allows for the application of high electrical fields to the capillary tube, which in turn leads to narrow sample bands and shorter analysis times. The capillary tubes are preferably made of fused silica and have a generally circular cross section, although capillary tubes having rectangular cross sections are available and can be used in the disclosed apparatus. The external surface of the capillary tubes can be coated with a material that imparts enhanced durability to the capillary tubes. Materials frequently used to impart enhanced durability to the capillary tube include, but are not limited to, polymer coatings such as polyimide, polytetrafluoroethylene (Teflon™), and the like. The effective length of the capillary tubes is dictated by the desired application and resolution.

FIG. 1 further shows a second high voltage power supply (16) that is connected to a second high voltage electrode (18). The second high voltage electrode (18) is electrically conductive. An operation configuration can be achieved when the second high voltage electrode (18) and a first buffer channel (20) are submerged in a second buffer reservoir (22) to a depth sufficient to establish an electrical connection between the second high voltage power supply (16) and the first buffer channel (20). Alternatively, a flushing configuration can be achieved by removing the first buffer channel (20) from the second buffer reservoir (22) to flush a first four-way junction (24) to a waste reservoir (25) by applying a positive pressure to a first syringe (23). The first syringe (23) can have the same contents as the second buffer reservoir (22); however the contents of the first syringe (23) and the second buffer reservoir (22) can differ while maintaining overall compatibility with operation of the apparatus. The first buffer channel (20) can alternatively be removed from the second buffer reservoir (22) to introduce a component other than the fluid present in the second buffer reservoir (22) by applying negative pressure to the first syringe (23) while the first buffer channel (20) is in contact with the component to be introduced. Components that can be introduced via the first buffer channel (20) include, but are not limited to, an internal standard, a binding agent, an enzyme, a labeling agent, and the like. After introducing the component other than the fluid present in the second buffer reservoir (22) into the first buffer channel (20), the first buffer channel (20) can be placed into the second buffer reservoir (22) for introduction of the component other than the fluid present in the second buffer reservoir (22) into the first four-way junction (24). As used herein a channel is any kind of fluid connection. In the preferred embodiment the channel is a fluid connection that is itself not open to the atmosphere even if the reservoir is open to the atmosphere; for example, a tube. In one embodiment, a pool of polynucleotides can be introduced into the first buffer channel (20) by submerging the first buffer channel (20) in a solution of a pool of polynucleotides and applying a negative pressure to the first syringe (23). The first buffer channel (20) delivers fluid from the second buffer reservoir (22) to a first four-way junction (24). The first four-way junction (24) fluidly connects the second end of the first capillary tube (11), the first buffer channel (20), a first syringe channel (26), and the first end of the second capillary tube (27). The second capillary tube (28) is further connected to a second four-way junction (30). The second four-way junction (30) fluidly connects the second end of the second capillary tube (29), a second buffer channel (32), a second syringe channel (34), and the first end of the third capillary tube (35). A third high voltage power supply (38) is connected to a third high voltage electrode (40). The third high voltage electrode (40) is electrically conductive. The third high voltage electrode (40) and the second buffer channel (32) are submerged in a third buffer reservoir (42) to a depth sufficient to establish an electrical connection between the third high voltage power supply (38) and the second buffer channel (32). The second buffer channel (32) can be removed from the third buffer reservoir (42) to flush the second four-way junction (30) to the waste reservoir (25) by applying a positive pressure to a second syringe (43). The second buffer channel (32) can alternatively be removed from the third buffer reservoir (42) to introduce a component other than the fluid present in the third buffer reservoir (42) by applying a negative pressure to the second syringe (43) while the second buffer channel (32) is in contact with the component to be introduced. The second buffer channel (32) delivers fluid from the third buffer reservoir (42) to the second four-way junction (30).

Generally, as used herein, a power supply is capable of supplying a direct current (DC) voltage that is then applied across the length of a capillary tube. The power supplies employed in the embodiment shown in FIG. 1 are high voltage power supplies capable of providing up to 30 kV DC and currents up to 300 μA. Choosing a power supply is largely dictated by the length of the capillary tube the voltage will be applied to and the desired volts-per-centimeter across the capillary tube. Separations can be performed using a wide range of volts-per-centimeter values including, but not limited to, 25 V/cm or greater. While the high voltage power supplies described herein are capable of providing up to 30 kV DC, one of skill in the art will recognize that other power supplies would be suitable. It is preferable for the power supplies to be capable of operation independent of one another.

The 2D-CE-SELEX apparatus of FIG. 1 terminates in a fraction collector. The third capillary tube (36) passes through a first port of a “T” fitting (44) and a second port of the “T” fitting (46). The second port of the “T” fitting (46) can be connected to a nozzle (48). The third capillary tube (36) can terminate within the nozzle (48), or the third capillary tube (36) can terminate outside of the end of the nozzle (48) which is distal to the “T” fitting (46). The nozzle (48) can be made of an electrically conductive material and can be further connected to a grounding wire (50) that itself can be connected to an electrical ground such as a common ground or an earth ground. The nozzle (48) can be made of a material that is not electrically conductive, provided that the second end of the third capillary tube (56) can be grounded in some other fashion. For example, an alternative grounding setup for the second end of the third capillary tube (56) can be to ground the sheath liquid within a sheath liquid reservoir (62). The grounding wire (50) must be electrically conductive. A third port of the “T” fitting (52) can supply a sheath liquid which enters the “T” fitting (54) and passes to the nozzle (48) where the sheath liquid ensheaths the second end of the third capillary tube (56). A computer controlled valve (58) that controls the flow of sheath liquid to the “T” fitting (54) can be connected to the third port of the “T” fitting (52). The sheath liquid is supplied by a length of tubing (60) from the sheath liquid reservoir (62). The length of tubing (60) can be made of plastic, metal, or another suitable material. The length of tubing (60) serves the purpose of being a transport path for the sheath liquid and should be chemically inert with respect to the sheath liquid. The sheath liquid reservoir (62) can be sealed and held under pressure by a compressed gas cylinder (64). The gas pressure released by the compressed gas cylinder (64) can be controlled by a regulator (73). The sheath liquid can be transported, under pressure, through the length of tubing (60), and passes through an inline filter (66) before reaching the computer controlled valve (58). When the computer controlled valve (58) is in the open position sheath liquid passes into the “T” fitting (54), displacing sheath liquid already present in the nozzle (48) and depositing fluid that has migrated from the second end of the third capillary tube (56) into a collection vessel, such as a well of a 96-well plate (68). The collection vessel, such as the 96-well plate (68), can be attached to an X-and-Y translational stage (70). The translational stage can also have mobility in the Z-axis. Alternatively, the nozzle (48) can be the component having mobility in the Z-axis. Yet another alternative would be to have the nozzle (48) translate in the X, Y, and Z-axis or any combination thereof.

Adjacent ends of the capillary tubes of the present disclosure can be aligned in a substantially co-axial manner. Similarly, the first buffer channel (20) and the first syringe channel (26) can be aligned in a substantially co-axial manner. The second buffer channel (32) and the second syringe channel (34) can also be aligned in a substantially co-axial manner. The first buffer channel (20) and the first syringe channel (26) can be parallel to the second buffer channel (32) and the second syringe channel (34). The first buffer channel (20) and the first syringe channel (26) can both be perpendicular to the directionality of the capillary tubes. The second buffer channel (32) and the second syringe channel (34) can both be perpendicular to the directionality of the capillary tubes. While the specific orientations disclosed here are co-axial and parallel with regard to the four-way junctions, one of skill in the art will appreciate that other orientations can be employed. It is preferred for the capillary tubes to be aligned in a substantially co-axial manner; however the buffer channels and their respective syringe channels can be aligned in a manner that is not substantially co-axial. That is, the four-way junctions can situate the buffer and syringe channels in such a way as to not be perpendicular to the capillary tubes. The first end of the first capillary tube (10), the second end of the third capillary tube (56), the second buffer reservoir (22), and the third buffer reservoir (42) can be kept at approximately the same level within the apparatus so as to minimize any syphoning that can occur due to differential heights of the components.

While a preferred embodiment (e.g. as shown in FIG. 1) uses a first syringe (23) and a second syringe (43) for flushing their respective four-way junctions, one of skill in the art will appreciate that the syringes can be replaced with any element that would accomplish the same ends. That is, the four-way junctions can be flushed and sample introduced at those points in the apparatus by other methods which include, but are not limited to, varying the heights of the respective buffer and syringe channels to use a gravimetric driving force rather than a mechanical driving force or sealing the respective buffer reservoirs and applying a differential pressure on the four-way junction.

A computer program can be used to operate the 2D-CE-SELEX apparatus. The computer program can control variables including the position of the X-and-Y translational stage (70), how long the computer controlled valve (58) is in the open position, at what interval the computer controlled valve (58) is cycled between open and closed, the operation of the first high voltage power supply (2), the operation of the second high voltage power supply (16), the operation of the third high voltage power supply (38), the duty cycle of the three high voltage power supplies, and the operation of the injector (14).

A target can be as small as a single ion or as large as an entire organism/cell; one of skill in the art will recognize that this range is not intended to be limiting but rather to demonstrate that targets vary widely based upon application. Some examples of targets that have been used include, but are not limited to, small organic molecules, proteins, whole prokaryotic and eukaryotic cells, peptides, and tissue. For example, a target could be designated as such because it is a marker for disease or a marker tied to a patient's prognosis. Another example as to why a target could be designated as such is that the target is present in a complex mixture and a desire exists to quantify that target in the presence of the complex mixture or there is a desire to purify the target from the complex mixture. The target need not be identified prior to binding ligands to the target; this can be referred to as de novo polynucleotide binding. It is possible to have identified an interesting characteristic without understanding the reason the characteristic exists, at which point it can be desirable to elucidate the reason the characteristic exists. One way the interesting characteristic can be investigated to understand the reason the characteristic exists is to identify polynucleotides that have an effect on the characteristic. The term target generally refers to an analyte for which there is an interest in identifying polynucleotides capable of binding either reversibly or irreversibly. A single target can be purified from a mixture of small organic molecules, proteins, whole prokaryotic and eukaryotic cells, peptides, and tissue. A mixture of small organic molecules, proteins, whole prokaryotic and eukaryotic cells, peptides, and tissue can include more than one target.

The 2D-CE-SELEX apparatus can be used for binding polynucleotides to targets and separating target-bound polynucleotides from polynucleotides that are not bound to a target. The first capillary tube (12) can be used to separate multiple targets from one another or to separate a target from other sample components that may interfere with polynucleotide binding. The target or targets can be introduced into the first capillary tube (12) by an injector (14) that can be present in the injection block (4). The target or targets can then be separated and moved through the first capillary tube (12) as a result of a voltage applied to the first buffer reservoir (8) by the first high voltage power supply (2). The voltage can be transmitted from the first high voltage power supply (2) to the first buffer reservoir (8) by the first high voltage electrode (6).

The polynucleotides that have been used in the present disclosure can be used as ligands. Ligands bind to targets with high affinity and specificity and are not limited to polynucleotides. One of skill in the art will appreciate that other ligands can be used including, but not limited to, antibodies and peptides. The introduction of the ligands to the apparatus can be achieved by submerging the first buffer channel (20) in a solution containing the ligands and applying a negative pressure to the first syringe channel (26) by pulling back on the plunger of the first syringe (23). One of skill in the art will appreciate that the method of introducing the ligands to the first buffer channel (20) is a matter of design and other methods of introducing the ligands can be employed, including, but not limited to, the use of an auto-sampler with pressure or electrokinetic introduction of the ligands. Once the ligands are introduced into the first buffer channel (20) the first buffer channel (20) can be submerged in the second buffer reservoir (22) to a depth sufficient to establish an electrical connection. To introduce the ligands to the sample flow within the capillary tubes a voltage can be applied to the second buffer reservoir (22) that results in a voltage drop across the first buffer channel (20). Other methods of introducing supplemental reagents have been described elsewhere (U.S. Pat. Nos. 4,936,974 and 5,798,032) and include using gravity or pressure.

The target can be introduced to the ligands upon transfer from the first capillary tube (12), through the first four-way junction (24), and into the second capillary tube (28). Once the target and ligands are co-localized within the second capillary tube (28) any ligands that are present within the second capillary tube (28) and have an affinity for the target will interact with the target. The contents of the second capillary tube (28) can be mixed in various ways. For example, the contents of the second capillary tube (28) can be mixed by diffusion. Generally, a mixing step can be deemed satisfactory if relative homogeneity is achieved.

After spending a period of time within the second capillary tube (28) the ligand-target mixture can be transferred from the second capillary tube (28), through the second four-way junction (30), and into the third capillary tube (36). Once within the third capillary tube (36) the ligand-target mixture can be separated into at least two populations. After separation on the third capillary tube (36) the separated ligand-target mixture passes through a nozzle (48) and can then be collected in a collection vessel, such as a 96-well plate (68). Other collection vessels can be used and one of skill in the art will recognize that the choice of a collection vessel is largely dictated by the desired post-separation manipulation. The deposition into a collection vessel allows the separation achieved on the third capillary tube (36) to be maintained for optional further manipulation such as, without limitation, polynucleotide amplification by polymerase chain reaction, ligand-target identification, or sample purification.

Many modifications and variations of the present disclosure are possible in light of the above teachings and can be practiced otherwise than as specifically described while within the scope of the appended claims.

### EXAMPLES

### Example 1

### Operation of the 2D-CE-SELEX Apparatus in the Absence of Target

An apparatus of general configuration as in FIG. 1 was used in the experiment described in this example. There was no target used in the experiment described in this example. The ligand used in the experiment described in this example was a polynucleotide specific to the protein human a-thrombin. The buffer used throughout the apparatus was 10 mM sodium tetraborate, 10 mM HEPES. The overall objective of the experiment described in this example was to demonstrate the operation of the 2D-CE-SELEX apparatus. A successful operation was indicated by periodic peaks in an electropherogram that corresponded to the fluorescently labeled pool of polynucleotides and by a low background signal between the peaks that correspond to the fluorescently labeled pool of polynucleotides.

The polynucleotide used had the sequence GGTTGGTGTGGTTGG (SEQ ID NO: 1) and was purchased from Sigma-Aldrich (The Woodlands, Tex., USA). The polynucleotide was labelled at the 5′ end with a fluorophore and was used at a concentration of 10 nM. The polynucleotide was suspended in 10 mM sodium tetraborate, 10 mM HEPES. The polynucleotide was introduced to the 2D-CE-SELEX apparatus by the first buffer channel (20), through the first four-way junction (24), and eventually filled the first buffer channel (20) and the second capillary tube (28). Once the polynucleotide was present in the second capillary tube (28) operation of the 2D-CE-SELEX apparatus began. The three power supplies were operated on a binary duty cycle. The first mode of the binary duty cycle was a transfer mode. During the transfer mode the first high voltage power supply (2) was set to 16.5 kV, the second high voltage power supply (16) was set to10.5 kV, and the third high voltage power supply (38) was set to 10.0 kV. The time that the 2D-CE-SELEX apparatus spent in the transfer mode was 2 seconds for a given cycle. As a result, the transfer mode effected a voltage drop across each of the capillary tubes. The first capillary tube (12) was 30 cm in length, the second capillary tube (28) was 1.0 cm in length, and the third capillary tube (36) was 30 cm in length. During the transfer mode the polynucleotide was transferred from the second capillary tube (28) through the second four-way junction (30) and into the third capillary tube (36) in a fractionated fashion. Simultaneous to the transfer of the fraction of the polynucleotide into the third capillary tube (36) a subsequent polynucleotide fraction was transferred into the second capillary tube (28) at the first four-way junction (24).

The second mode of the binary duty cycle was a separation mode. During the separation mode the first high voltage power supply (2), the second high voltage power supply (16), and the third high voltage power supply (38) were set to 10.0 kV. The time that the 2D-CE-SELEX apparatus spent in the separation mode was 298 seconds for a given cycle. As a result, no electric field was present on the first capillary tube (12) and the second capillary tube (28) during the separation mode. However, due to the second end of the third capillary tube (56) being held at electrical ground, an electric field existed was present on the third capillary tube (36). During the separation mode a transferred polynucleotide fraction migrated toward the second end of the third capillary tube (56). Completion of a duty cycle is defined as a completion of both a transfer cycle and a separation cycle. That is, the duty cycle is 300 seconds as described in this example. Upon the completion of one duty cycle a subsequent duty cycle began automatically. The cycling of the high voltage power supplies continued for 14 cycles and demonstrated the operation of the 2D-CE-SELEX apparatus in the absence of a target.

This experiment employed a laser-induced fluorescence detection assembly rather than the fraction collector to detect the fluorescently labelled polynucleotide. The laser-induced fluorescence detection assembly excited the fluorescent polynucleotide within a sheath flow cuvette using a CW 532 nm diode-pumped laser (CrystaLaser Model CL532-025). Fluorescence was collected at an angle of 90 degrees from the incident laser beam, passed through a bandpass filter, and was detected using a single-photon counting avalanche photodiode module (PerkinElmer, Montreal, PQ Canada).

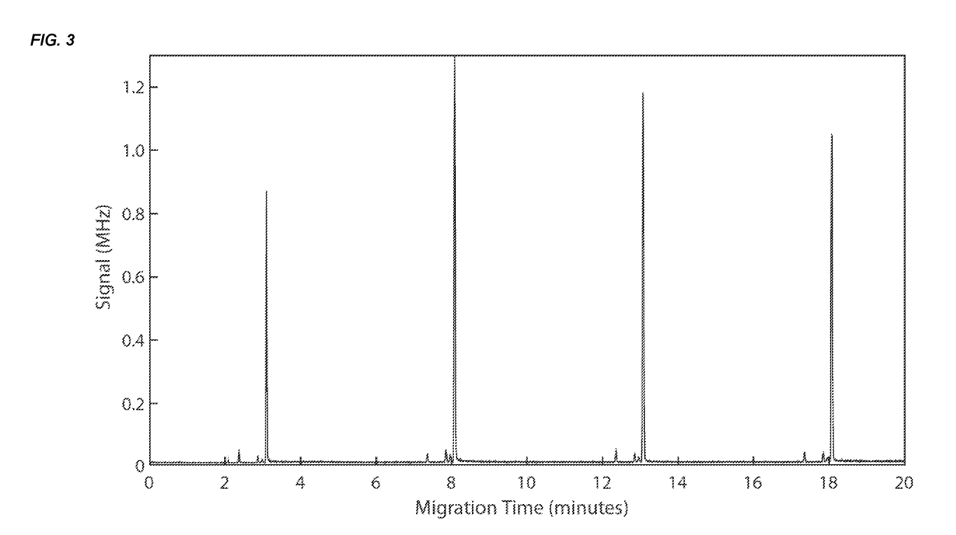

FIG. 3 shows a representative electropherogram of the results of this experiment. The electropherogram shows sharp, symmetrical peaks that correspond to the fluorescently labelled polynucleotide that are separated by a low baseline between the peaks. The time between peaks corresponds to the amount of time that only the third high voltage power supply (38) was effecting a separation and the other high voltage power supplies were applying the same voltage such that no voltage drop was present across the first capillary tube (12) or the second capillary tube (28). There are a few low intensity peaks (e.g. height of 5,000 Hz or less) that correspond to impurities in the polynucleotide sample. The polynucleotide sample in this experiment had approximately 1 ppt impurities.

### Example 2

### Operation of the 2D-CE-SELEX Apparatus with a Single Target

An apparatus of general configuration as in FIG. 1 was used in the experiment described in this example. The target used in the experiment described in this example was the protein human α-thrombin (thrombin). The ligand used in the experiment described in this example was a polynucleotide specific to the protein human α-thrombin. The buffer used throughout the apparatus was 10 mM sodium tetraborate, 10 mM HEPES. The overall objective of the experiment described in this example was to demonstrate the 2D-CE-SELEX apparatus' ability to associate a target (thrombin) with a polynucleotide that is known to bind to the target. A successful operation was indicated by periodic peaks in an electropherogram that correspond to the fluorescently labeled polynucleotide, by the appearance of a significant peak that corresponds to the fluorescently labeled polynucleotide bound to thrombin, and by a low background signal between the peaks that correspond to the fluorescently labeled polynucleotide.

The polynucleotide used had the sequence GGTTGGTGTGGTTGG (SEQ ID NO: 1) and was purchased from Sigma-Aldrich (The Woodlands, Tex., USA). The polynucleotide was labelled at the 5′ end with a fluorophore and was used at a concentration of 100 nM. The polynucleotide was suspended in 10 mM sodium tetraborate, 10 mM HEPES. The polynucleotide entered the 2D-CE-SELEX apparatus by pulling back on the plunger of the first syringe (23) while the first buffer channel (20) was submerged in a solution of 100 nM polynucleotide. After the polynucleotide was introduced to the first buffer channel (20), the first buffer channel (20) was placed in the second buffer reservoir (22). The target, thrombin, was suspended in 10 mM sodium tetraborate, 10 mM HEPES at a concentration of 220 μg/mL. The first capillary tube (12) was filled entirely with the suspended thrombin. Thrombin was introduced into the first capillary tube (12) by an injector (14) that was present in the injection block (4). Thrombin was continuously supplied to the first capillary tube (12) via the injection block (4) throughout the experiment. The polynucleotide introduced to the first buffer channel (20) supplied the 2D-CE-SELEX apparatus with polynucleotide for the duration of the experiment.

Once the 2D-CE-SELEX apparatus had been prepared by introducing thrombin and the polynucleotide a voltage was applied across each capillary tube such that the thrombin would progress toward the first four-way junction (24) and the polynucleotide would fill the second capillary tube (28). The voltage applied across the first capillary tube (12) was 200 V/cm. The voltage applied across the second capillary tube (28) was 500 V/cm. The voltage applied across the third capillary tube (36) was 500 V/cm. The stated voltages were used as a final preparation step to fully prepare the 2D-CE-SELEX apparatus for the experiment.

The first buffer reservoir (8) was a plastic tube that had been trimmed to fit within a bottom portion of the injection block (4) and was capable of holding approximately 100 μL of fluid. The first buffer reservoir (8) was sealed when the bottom portion of the injection block was attached to the rest of the injection block (4). The second buffer reservoir (22) and the third buffer reservoir (42) were plastic tubes capable of holding approximately 1.5 mL, although larger or smaller volume reservoirs can be employed, and were not sealed but remained open to atmospheric pressure. Each of the buffer reservoirs were filled with 10 mM sodium tetraborate, 10 mM HEPES.

Once the polynucleotide was present in the second capillary tube (28) and thrombin was present in the first capillary tube (12) operation of the 2D-CE-SELEX apparatus began. The three power supplies were operated on a binary duty cycle. The binary duty cycle consisted of two modes. The first mode of the binary duty cycle was a transfer mode. During the transfer mode the first high voltage power supply (2) was set to 16.5 kV, the second high voltage power supply (16) was set to10.5 kV, and the third high voltage power supply (38) was set to 10.0 kV. The time that the 2D-CE-SELEX apparatus spent in the transfer mode was 2 seconds for a given cycle. As a result, an electric field was present on each of the capillary tubes during the transfer mode. The first capillary tube (12) was 30 cm in length, the second capillary tube (28) was 1.0 cm in length, and the third capillary tube (36) was 30 cm in length. During the transfer mode the polynucleotide was transferred from the second capillary tube (28) through the second four-way junction (30) and into the third capillary tube (36) in a fractionated fashion. Similarly, thrombin was transferred from the first capillary tube (12) through the first four-way junction (24) and into the second capillary tube (28) in a fractionated fashion.

The second mode of the binary duty cycle was a separation mode. During the separation mode the first high voltage power supply (2), the second high voltage power supply (16), and the third high voltage power supply (38) were set to the 10.0 kV. The time that the 2D-CE-SELEX apparatus spent in the separation mode was 298 seconds for a given cycle. As a result, no electric field was present on the first capillary tube (12) and the second capillary tube (28) during the separation mode. However, due to the second end of the third capillary tube (56) being held at electrical ground, an electric field was present on the third capillary tube (36). While in the second capillary tube (28), thrombin was allowed to mix with the polynucleotide by diffusion. After thrombin had interacted with the polynucleotide in the second capillary tube (28) the contents of the second capillary tube (28) comprised a sample mixture. Completion of a duty cycle is defined as a completion of a transfer cycle and a separation cycle. That is, the duty cycle is 300 seconds as described in this example. Upon the completion of one duty cycle a subsequent duty cycle began automatically. The sample mixture was transferred from the second capillary tube (28) through the second four-way junction (20) and into the third capillary tube (36) in a fractionated fashion. Once in the third capillary tube (36) the sample mixture was separated into two detectable populations. The first detectable population was polynucleotide that had bound to thrombin and the second detectable population was polynucleotide that had not bound to thrombin. The separation was based on the size-to-charge ratio of the sample mixture components.

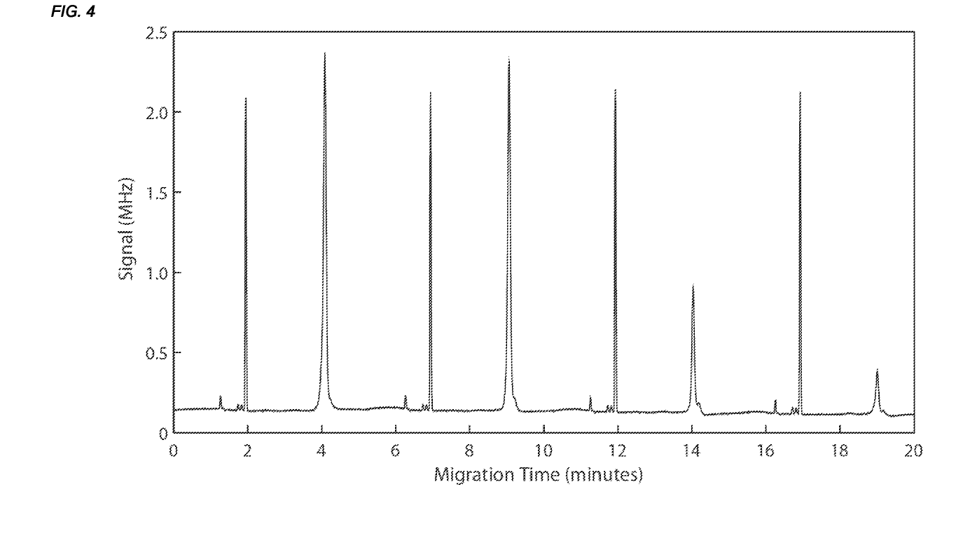

The cycling of the high voltage power supplies continued until sufficient cycles had been performed to demonstrate the operation of the 2D-CE-SELEX apparatus with a single target. Sufficient cycles to demonstrate the operation of the 2D-CE-SELEX apparatus with a single target was determined by the appearance of a second, significant peak that did not correspond to impurity peaks. The second, significant peak can be seen in FIG. 4 and decreases in intensity as a function of duty cycle number. The decrease of the intensity of the peak was attributed to a decrease in thrombin-bound polynucleotides. This experiment employed a laser-induced fluorescence detection assembly to detect the fluorescently labelled polynucleotide rather than the fraction collector. The laser-induced fluorescence detection assembly excited the fluorescent polynucleotide within a sheath flow cuvette using a CW 532 nm diode-pumped laser (CrystaLaser Model CL532-025). Fluorescence was collected at an angle of 90 degrees from the incident laser beam, passed through a bandpass filter, and was detected using a single-photon counting avalanche photodiode module (PerkinElmer, Montreal, PQ Canada).

The voltage that was applied to the third high voltage power supply (38) was transmitted from the third high voltage power supply (38) to the third buffer reservoir (42) by the third high voltage electrode (40). The voltage applied to the third buffer reservoir (42) was transmitted to the second four-way junction (30) by the second buffer channel (32) that was submerged in the third buffer reservoir (42).

A representative electropherogram showing separation cycles that lacked a peak that corresponded to a polynucleotide-thrombin complex is shown in FIG. 3. A representative electropherogram showing separation cycles that contained a peak that corresponded to a polynucleotide-thrombin complex is shown in FIG. 4. At the time of performing the experiment it was believed that the second, significant peak was attributable to the polynucleotide-thrombin complex. Further investigation showed the second, significant peak was not attributable to the polynucleotide-thrombin complex in this experiment. However, upon the binding of a polynucleotide to a target a change in migration time is observed when performing a separation based on the size-to-charge-ratio of the analytes.

### Example 3

### Operation of the 2D-CE-SELEX Apparatus with Multiple Targets Present

An apparatus of general configuration as in FIG. 1 is used in the experiment described in this example. Multiple targets are used in the experiment described in this example. The ligand used in the experiment described in this example is a pool of polynucleotides. The pool of polynucleotides consist of a central random sequence region that is flanked on either side by regions of known sequence that are of polymerase chain reaction primer length and capable of being used to amplify the central random sequence region. The buffer used throughout the apparatus is a buffer compatible with polynucleotides binding to targets. The overall objective of the experiment described in this example is to demonstrate the 2D-CE-SELEX apparatus' ability to associate multiple targets with a polynucleotide or polynucleotides. A successful operation is indicated by periodic peaks in an electropherogram that correspond to the polynucleotide, by the appearance of a significant peak that corresponds to the polynucleotide or polynucleotides bound to a target, and by a low background signal between the peaks that correspond to the polynucleotide or polynucleotides.

The pool of polynucleotides consists of a 40 base pair central random sequence region that is flanked on either side by 20 base pair regions of known sequence that are of polymerase chain reaction primer length and capable of being used to amplify the central random sequence region. The primer on the 5′ end of the pool of polynucleotides has the following sequence AGCAGCACAGAGGTCAGATG (SEQ ID NO: 2). While a specific primer and sequence are used for the 5′ primer in the present example, other sequences can be used. The primer on the 3′ end of the pool of polynucleotides has the following sequence CCTATGCGTGCTACCGTGAA (SEQ ID NO: 3). While a specific primer and sequence are used for the 3′ primer in the present example, other sequences can be used. The pool of polynucleotides has the following general composition AGCAGCACAGAGGTCAGATG (SEQ ID NO: 2)-N(40)-CCTATGCGTGCTACCGTGAA (SEQ ID NO: 3), where N denotes a random base. The pool of polynucleotides is suspended in a buffer compatible with polynucleotides binding to targets. The pool of polynucleotides enter the 2D-CE-SELEX apparatus by pulling back on the plunger of the first syringe (23) while the first buffer channel (20) is submerged in a pool of polynucleotides solution having a concentration of at least 1 nM. After the pool of polynucleotides is introduced to the first buffer channel (20), the first buffer channel (20) is placed in the second buffer reservoir (22). The targets are suspended in a buffer compatible with electrophoresis and are injected onto the first capillary tube (12) by the injector (14) that is present in the injection block (4). After injection of the multiple targets onto the first capillary tube (12) the first buffer reservoir (8) contains the buffer used to suspend the multiple targets. The pool of polynucleotides introduced to the first buffer channel (20) supplies the 2D-CE-SELEX apparatus with polynucleotides for the duration of the experiment.

Once the pool of polynucleotides is present in the second capillary tube (28) and the multiple targets are present in the first capillary tube (12) operation of the 2D-CE-SELEX apparatus begins. The three power supplies are operated on a binary duty cycle. The binary duty cycle consists of two modes. The first mode of the binary duty cycle is a transfer mode. During the transfer mode the first high voltage power supply (2) is set to a voltage higher in magnitude than the second high voltage power supply (16), the second high voltage power supply (16) is set to a voltage higher in magnitude than the third high voltage power supply (38), and the third high voltage power supply (38) is set to a voltage higher in magnitude than zero. The transfer mode effects a voltage drop across each of the capillary tubes. During the transfer mode the pool of polynucleotides is transferred from the second capillary tube (28) through the second four-way junction (30) and into the third capillary tube (36) in a fractionated fashion. Similarly, the multiple targets are transferred from the first capillary tube (12) through the first four-way junction (24) and into the second capillary tube (28) in a fractionated fashion after they have traversed the length of the first capillary tube (12).

The second mode of the binary duty cycle is a separation mode. During the separation mode the first high voltage power supply (2), the second high voltage power supply (16), and the third high voltage power supply (38) are set to the same voltage. As a result, the separation mode effects no voltage drop across the first capillary tube (12) and the second capillary tube (28). However, due to the second end of the third capillary tube (56) being held at electrical ground a voltage drop exists across the length of the third capillary tube (36). While in the second capillary tube (28), the targets interact with the pool of polynucleotides. After the targets have interacted with the pool of polynucleotides in the second capillary tube (28) the contents of the second capillary tube (28) comprise a sample mixture. Completion of a duty cycle is defined as a completion of a transfer cycle and a separation cycle. That is, the duty cycle is one repetition of the binary cycles. Upon the completion of one duty cycle a subsequent duty cycle begins automatically. The sample mixture is transferred from the second capillary tube (28) through the second four-way junction (20) and into the third capillary tube (36) in a fractionated fashion. Once in the third capillary tube (36) the sample mixture is separated into two detectable populations. The first detectable population is polynucleotide that has bound to a target and the second detectable population is polynucleotide that has not bound to a target. The separation is based on the size-to-charge ratio of the sample mixture components. The time that the 2D-CE-SELEX apparatus spends in the transfer mode is shorter than the time that the 2D-CE-SELEX apparatus spends in the separation mode for a given cycle.

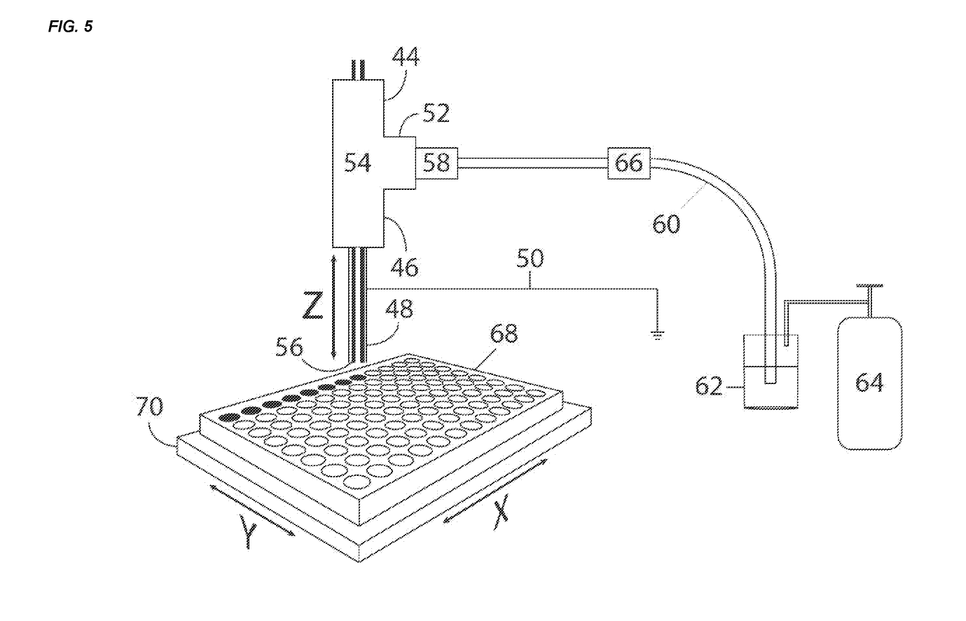

The cycling of the high voltage power supplies continues until sufficient cycles have been performed to expose each of the multiple targets to the pool of polynucleotides and separate the resultant sample mixture. Sufficient cycles to expose each of the multiple targets to the pool of polynucleotides and separate the resultant sample mixture is evidenced by the disappearance of peaks that correspond to polynucleotide bound to a target. This experiment employs a fraction collector as in FIG. 5 to collect, and maintain the separation of, separated sample mixture components.

The voltage that is applied to the third high voltage power supply (38) is transmitted from the third high voltage power supply (38) to the third buffer reservoir (42) by the third high voltage electrode (40). The voltage applied to the third buffer reservoir (42) is transmitted to the second four-way junction (30) by the second buffer channel (32) that is submerged in the third buffer reservoir (42).

The first buffer reservoir (8) is a plastic tube that has been trimmed to fit within a bottom portion of the injection block (4) and is capable of holding approximately 100 μL of fluid, although larger or smaller volume reservoirs can be employed. The first buffer reservoir (8) is sealed when the bottom portion of the injection block is attached to the rest of the injection block (4). The second buffer reservoir (22) and the third buffer reservoir (42) are plastic tubes capable of holding approximately 1.5 mL, although larger or smaller volume reservoirs can be employed, and are not sealed but remained open to atmospheric pressure. Each of the buffer reservoirs are filled with a buffer compatible with electrophoresis.

Polynucleotide-target mixture that migrates to the second end of the third capillary tube (56) is deposited into a collection vessel, such as a 96-well plate (68), by a sheath liquid that is controlled by a computer controlled valve (58). The sheath liquid reservoir (62) is made of glass, sealed, and held under pressure from a compressed gas cylinder (64). The deposition into a collection vessel allows the separation achieved on the third capillary tube (36) to be maintained for optional further manipulation such as, without limitation, polynucleotide amplification by polymerase chain reaction, polynucleotide-target identification, or sample purification.

The pool of polynucleotides is introduced by the first buffer channel (20), through the first four-way junction (24), and is exposed to one of the targets when the target is transferred from the first capillary tube (12) to the second capillary tube (28). Within the second capillary tube (28) the target and the pool of polynucleotides are allowed a period of time to interact. Following the transfer to and interaction within the second capillary tube (28) the polynucleotide-target mixture is transferred from the second capillary tube (28) through the second four-way junction (30) and into the third capillary tube (36). The transfer from the second capillary tube (28) through the second four-way junction (30) and into the third capillary tube (36) is effected by applying a lower voltage to the third high voltage power supply (38) relative to the second high voltage power supply (16). The lower voltage that is applied to the third high voltage power supply (38) is transmitted from the third high voltage power supply (38) to the third buffer reservoir (42) by the third high voltage electrode (40). The voltage applied to the third buffer reservoir (42) is transmitted to the second four-way junction (30) by the second buffer channel (32) that is submerged in the third buffer reservoir (42).

### Example 4

### Operation of the 2D-CE-SELEX Apparatus with a Target and Impurities

An apparatus of general configuration as in FIG. 1 is used in the experiment described in this example. A target solution containing impurities is used in the experiment described in this example. The ligand used in the experiment described in this example is a pool of polynucleotides. The pool of polynucleotides consist of a central random sequence region that is flanked on either side by regions of known sequence that are of polymerase chain reaction primer length and capable of being used to amplify the central random sequence region. The buffer used throughout the apparatus is a buffer compatible with polynucleotides binding to targets. The overall objective of the experiment described in this example is to demonstrate the 2D-CE-SELEX apparatus' ability to separate a target from impurities present that can interfere with polynucleotide binding and to associate the separated target with a polynucleotide or polynucleotides. A successful operation is indicated by periodic peaks in an electropherogram that correspond to the polynucleotide, by the appearance of a significant peak that corresponds to the polynucleotide or polynucleotides bound to a target, and by a low background signal between the peaks that correspond to the polynucleotide or polynucleotides.

The pool of polynucleotides consists of a 40 base pair central random sequence region that is flanked on either side by 20 base pair regions of known sequence that are of polymerase chain reaction primer length and capable of being used to amplify the central random sequence region. The pool of polynucleotides has the following general composition AGCAGCACAGAGGTCAGATG (SEQ ID NO: 2)-N(40)-CCTATGCGTGCTACCGTGAA (SEQ ID NO: 3), where N denotes a random base. The pool of polynucleotides is suspended in a buffer compatible with polynucleotides binding to targets. The pool of polynucleotides enter the 2D-CE-SELEX apparatus by pulling back on the plunger of the first syringe (23) while the first buffer channel (20) is submerged in a pool of polynucleotides solution having a concentration of at least 1 nM. After the pool of polynucleotides is introduced to the first buffer channel (20), the first buffer channel (20) is placed in the second buffer reservoir (22). The target solution containing impurities is suspended in a buffer compatible with electrophoresis and is injected onto the first capillary tube (12) by the injector (14) that is present in the injection block (4). After injection of the target solution containing impurities onto the first capillary tube (12) the first buffer reservoir (8) contains the buffer used to suspend the target solution containing impurities. The pool of polynucleotides introduced to the first buffer channel (20) supplies the 2D-CE-SELEX apparatus with polynucleotides for the duration of the experiment.

Once the pool of polynucleotides is present in the second capillary tube (28) and the target solution containing impurities is present in the first capillary tube (12) operation of the 2D-CE-SELEX apparatus begins. The three power supplies are operated on a binary duty cycle. The binary duty cycle consisted of two modes. The first mode of the binary duty cycle is a transfer mode. During the transfer mode the first high voltage power supply (2) is set to a voltage higher in magnitude than the second high voltage power supply (16), the second high voltage power supply (16) is set to a voltage higher in magnitude than the third high voltage power supply (38), and the third high voltage power supply (38) is set to a voltage higher in magnitude than zero. The transfer mode effects a voltage drop across each of the capillary tubes. During the transfer mode the pool of polynucleotides is transferred from the second capillary tube (28) through the second four-way junction (30) and into the third capillary tube (36) in a fractionated fashion. Similarly, after the target solution containing impurities has been separated on the first capillary tube (12) and traversed the length of the first capillary tube (12) the separated target is transferred from the first capillary tube (12) through the first four-way junction (24) and into the second capillary tube (28) in a fractionated fashion during the transfer mode.

The second mode of the binary duty cycle is a separation mode. During the separation mode the first high voltage power supply (2), the second high voltage power supply (16), and the third high voltage power supply (38) are set to the same voltage. As a result, the separation mode effects no voltage drop across the first capillary tube (12) and the second capillary tube (28). However, due to the second end of the third capillary tube (56) being held at electrical ground a voltage drop exists across the length of the third capillary tube (36). While in the second capillary tube (28), the target interacts with the pool of polynucleotides. After the target has interacted with the pool of polynucleotides in the second capillary tube (28) the contents of the second capillary tube (28) comprise a sample mixture. Completion of a duty cycle is defined as a completion of a transfer cycle and a separation cycle. That is, the duty cycle is one repetition of the binary cycles. Upon the completion of one duty cycle a subsequent duty cycle begins automatically. The sample mixture is transferred from the second capillary tube (28) through the second four-way junction (20) and into the third capillary tube (36) in a fractionated fashion. The term fractionated fashion is intended to describe the transfer of the sample mixture from one capillary tube to another in such a way that the entire sample mixture is not transferred at once. Instead, the entire sample mixture is transferred from one capillary to another over the course of more than one binary cycle. Once in the third capillary tube (36) the sample mixture is separated into two detectable populations. The first detectable population is polynucleotide that has bound to a target and the second detectable population is polynucleotide that has not bound to a target. The separation is based on the size-to-charge ratio of the sample mixture components. The time that the 2D-CE-SELEX apparatus spends in the transfer mode is shorter than the time that the 2D-CE-SELEX apparatus spends in the separation mode for a given cycle.

The cycling of the high voltage power supplies continues until sufficient cycles have been performed to expose all of the target molecules to the pool of polynucleotides and separate the resultant sample mixture. Sufficient cycles to expose all of the target molecules to the pool of polynucleotides and separate the resultant sample mixture is evidenced by the disappearance of peaks that correspond to polynucleotide bound to a target. This experiment employs a fraction collector as in FIG. 5 to collect, and maintain the separation of, separated sample mixture components.

The voltage that is applied to the third high voltage power supply (38) is transmitted from the third high voltage power supply (38) to the third buffer reservoir (42) by the third high voltage electrode (40). The voltage applied to the third buffer reservoir (42) is transmitted to the second four-way junction (30) by the second buffer channel (32) that is submerged in the third buffer reservoir (42).

The first buffer reservoir (8) is a plastic tube that has been trimmed to fit within a bottom portion of the injection block (4) and is capable of holding approximately 100 μL of fluid, although larger or smaller volume reservoirs can be employed. The first buffer reservoir (8) is sealed when the bottom portion of the injection block is attached to the rest of the injection block (4). The second buffer reservoir (22) and the third buffer reservoir (42) are plastic tubes capable of holding approximately 1.5 mL, although larger or smaller volume reservoirs can be employed, and are not sealed but remained open to atmospheric pressure. Each of the buffer reservoirs are filled with a buffer compatible with electrophoresis.

Polynucleotide-target mixture that migrates to the second end of the third capillary tube (56) is deposited into a collection vessel, such as a 96-well plate (68), by a sheath liquid that is controlled by a computer controlled valve (58). The sheath liquid reservoir (62) is made of glass, sealed, and held under pressure from a compressed gas cylinder (64). The deposition into a collection vessel allows the separation achieved on the third capillary tube (36) to be maintained for optional further manipulation such as, without limitation, polynucleotide amplification by polymerase chain reaction, polynucleotide-target identification, or sample purification.

### Example 5

### Operation of the Fraction Collector with a Single Capillary Tube

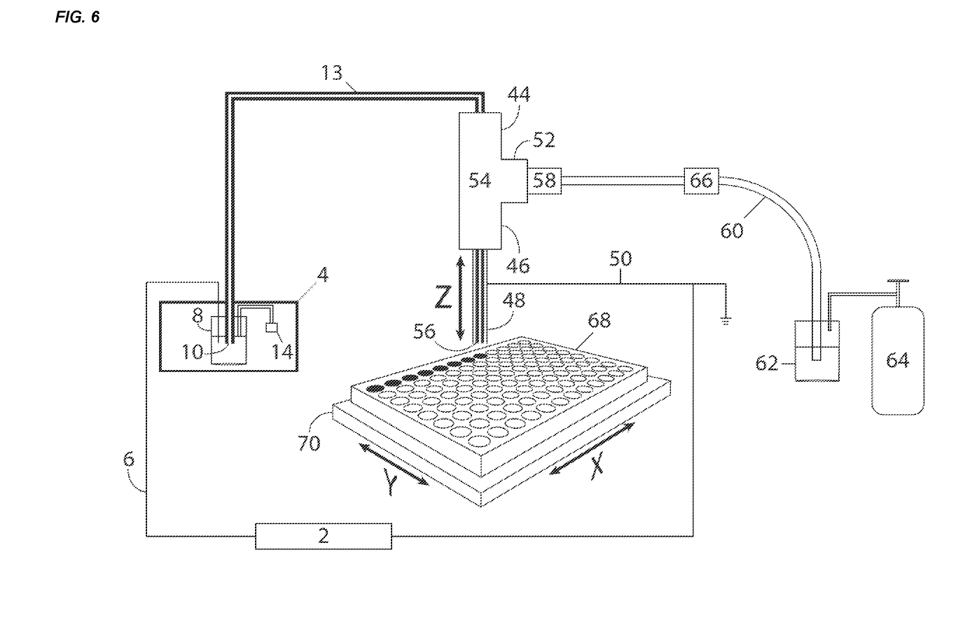

An apparatus of general configuration as in FIG. 6 was used in the experiment described in this example. A target solution containing human α-thrombin (thrombin) was used in the experiment described in this example. The ligand used in the experiment described in this example was a pool of polynucleotides. The pool of polynucleotides consisted of a central random sequence region that was flanked on either side by regions of known sequence that were of polymerase chain reaction primer length and capable of being used to amplify the central random sequence region. The pool of polynucleotides had the following composition 5′-AGCAGCACAGAGGTCAGATG (SEQ ID NO: 2)-N(40)-CCTATGCGTGCTACCGTGAA (SEQ ID NO: 3) -3′, where N denotes a random nucleotide. Two pools of polynucleotides of the same composition as stated were used. The only difference between the two pools of polynucleotides was that one of the pools of polynucleotides had a fluorescent label attached to the 5′ end while the other pool of polynucleotides had no fluorescent label. The fluorescently labeled pool of polynucleotides was used to generate reference electropherograms with a laser-induced fluorescence detection assembly rather than a fraction collector. The laser-induced fluorescence detection assembly excited the fluorescent polynucleotide within a sheath flow cuvette using a CW 532 nm diode-pumped laser (CrystaLaser Model CL532-025). Fluorescence was collected at an angle of 90 degrees from the incident laser beam, passed through a bandpass filter, and was detected using a single-photon counting avalanche photodiode module (PerkinElmer, Montreal, PQ Canada). The pool of polynucleotides that was not labeled with a fluorescent tag utilized the fraction collector assembly rather than the laser-induced fluorescence detection assembly. The fraction collector assembly used in this example is shown in FIG. 6.

The separation buffer used in the capillary tube (13) was a 10 mM sodium tetraborate, 10 mM HEPES buffer solution. The capillary tube (13) had a first end of the capillary tube (71) and a second end of the capillary tube (72). The sheath liquid reservoir (62) contained sheath buffer. The sheath buffer was 10 mM sodium tetraborate, 10 mM HEPES with real-time PCR reagents suspended in solution. To limit sample handling and sample loss, the sheath buffer contained all necessary real-time PCR reagents; reagents and final concentrations for 10 μL reactions were iTaq Universal SYBR Green Supermix (1×) and forward and reverse primers (300 nM each). The overall objective of the experiment described in this example was to demonstrate the ability of the fraction collector to be used for depositing fractions that had been separated on a single capillary tube. A successful operation was indicated by the appearance of peaks that migrated away from the bulk polynucleotide peak and were not present in laser-induced fluorescence based control electropherograms that only contained the pool of polynucleotides.

The pool of polynucleotides was injected for 5 seconds at 5 kV onto a preconditioned 45 cm capillary tube. The separation was performed at 15 kV. Hard-shell, white well, 96-well PCR plates (Bio-rad) were used for fraction collection. A fraction width of 7 seconds was determined by reference data obtained by the laser-induced fluorescence detection assembly. The valve pulse width was set to 0.05 seconds, precisely dispensing sheath buffer. Fraction collection and electrophoresis began simultaneously. A computer program written in LabVIEW software was used to control the operation of the fraction collector and the separation voltage simultaneously. A computer program was used to collect fluorescence data and control the separation voltage during control runs using the fluorescently labeled pool of polynucleotides.

After fraction collection, the 96-well PCR plate was sealed and centrifuged, bringing the deposited fractions to the bottom of the wells for amplification. The PCR protocol was designed to optimize the reaction based on the annealing temperatures of the forward, AGCAGCACAGAGGTCAGATG (SEQ ID NO: 4), and reverse, TTCACGGTAGCACGCATAGG (SEQ ID NO: 5), primers and the pre-mixed components of iTaq Universal SYBR Green Supermix (Bio-rad). The PCR protocol was 95° C. for 3 minutes followed by 40 cycles of 95° C. for 30 seconds (denature), 56.7° C. for 30 seconds (anneal), and 72° C. for 30 seconds (extend). Following each extension, real-time fluorescence was measured in each well using CFX96 Touch Real-Time PCR Detection System (Bio-rad).

A 10 μL aliquot of the pool of polynucleotides, at a concentration of 100 μM, was added to 10 μL of binding buffer (50 mM TRIS, 100 mM NaCl, 1 mM CaCl₂) and was heated to 94° C. to destroy secondary structures that can form during storage. The solution was cooled by 0.5° C./s to a final temperature of 20° C. in a thermal cycler (PTC-100, MJ Research). A heat-treated 10 μM solution of the pool of polynucleotides was incubated at room temperature with 1 mg/mL human α-thrombin protein (Hematologic Technologies, Inc., Vermont USA) for a minimum of 15 minutes to allow binding.

Fractions were collected and amplified. Post-amplification, wells containing thrombin were combined and submitted for deep sequencing. In preparation for deep sequencing analysis, an Illumina library was constructed on the submitted sample. Sample quality was verified via a bioanalyzer trace; the majority of the material present was 214 base pairs. Illumina's TruSeq Universal Adapter AATGATACGGCGACCACCGAGATCTACACTCTTTCCCTACACGACGCTCTTCCGATCT (SEQ ID NO: 6) and TruSeq Adapter, Index 6 GATCGGAAGAGCACACGTCTGAACTCCAGTCACGCCAATATCTCGTATGCCGTCTTCTGCTT G (SEQ ID NO: 7) were used. Sequencing was performed using a MiSeq nano flow cell for a single 140 base pair read, and the sample was spiked with 25% PhiX control to generate library diversity. The MiSeq run generated over 1 million high quality reads. The sequences were compared with established thrombin-binding polynucleotide sequence.

Preprocessing extracted roughly 800,000 reads from the original dataset of 1,082,975 sequences. Using Perl program code, the ligated adapter sequences were trimmed, followed by the priming regions. The desired sequences of lengths 38-42 bases, representing the central random region of the pool of polynucleotides, were selected for analysis; this subset encompasses approximately 98% of the high quality reads. In this experiment an assumption was made that 4 or more instances of neighboring guanines constitute an exact or near-exact match based on the three-dimensional structure of the thrombin-binding polynucleotide; ˜48,000 sequences contained four or more ‘GG’. The data were transformed into FASTA format and mapped against the established 15-mer GGTTGGTGTGGTTGG (SEQ ID NO: 1) and 29-mer AGTCCGTGGTAGGGCAGGTTGGGGTGACT (SEQ ID NO: 8) thrombin-binding polynucleotide sequences utilizing the Burrows-Wheeler alignment tool. Burrows-Wheeler alignment tool fastmap analysis returned 10,534 sequence matches to the 15-mer and 17,606 matches to the 29-mer, omitting duplicate matches found on a single read. MEME motif discovery tool (version 4.9.1) was also used to detect enriched sequences/motifs to confirm results.

What is claimed is:

1. An apparatus for identifying a polynucleotide capable of binding a target comprising:
a first high voltage power supply connected to a first high voltage electrode;
said first high voltage electrode inserted into an injection block comprising a first buffer reservoir;
a first end of a first capillary tube inserted into said injection block and submerged in said first buffer reservoir;
a second high voltage power supply connected to a second high voltage electrode submerged in a second buffer reservoir;
a third high voltage power supply connected to a third high voltage electrode submerged in a third buffer reservoir;
a first buffer channel and a first waste outlet;
a second capillary tube having a substantially co-axial alignment with said first capillary tube;
a first four-way junction between a second end of said first capillary tube, a first end of said second capillary tube, said first buffer channel, and said first waste outlet;
said first four-way junction being connected to said second buffer reservoir via said first buffer channel;
a second buffer channel and a second waste outlet;
a second four-way junction between a second end of said second capillary tube, a first end of a third capillary tube, said second buffer channel, and said second waste outlet;
said second four-way junction being connected to said third buffer reservoir via said second buffer channel;
wherein said injection block is fluidly attached to an injector capable of injecting fluid into said first end of said first capillary tube.

2. The apparatus for identifying a polynucleotide capable of binding a target of claim 1, wherein said first buffer channel and said first waste outlet are perpendicular to said first capillary tube and said second capillary tube.

3. The apparatus for identifying a polynucleotide capable of binding a target of claim 1, wherein said second buffer channel and said second waste outlet are perpendicular to said second capillary tube and said third capillary tube.

4. The apparatus for identifying a polynucleotide capable of binding a target of claim 1, wherein said second buffer channel and said second waste outlet are parallel to said first buffer channel and said first waste outlet.

5. The apparatus for identifying a polynucleotide capable of binding a target of claim 1, further comprising a fraction collector fluidly connected to a second end of said third capillary tube.

6. The apparatus for identifying a polynucleotide capable of binding a target of claim 5, further comprising a computer program capable of controlling said first high voltage power supply, said second high voltage power supply, said third high voltage power supply, said injector, and said fraction collector.

7. The apparatus for identifying a polynucleotide capable of binding a target of claim 1, wherein the first four-way junction is sealed.

8. The apparatus for identifying a polynucleotide capable of binding a target of claim 1, wherein the second four-way junction is sealed.

9. An apparatus for identifying a polynucleotide capable of binding a target comprising:
a first four-way junction, a second four-way junction, a first capillary tube, a second capillary tube, and a third capillary tube;
the first, second and third capillary tubes each having a first and a second end;
said first capillary tube, said second capillary tube, and said third capillary tube being aligned in a substantially co-axial alignment to one another;
said first end of said first capillary tube and said first end of said second capillary tube being fluidly attached at said first four-way junction, said second end of said second capillary tube and said first end of said third capillary tube being fluidly attached at said second four-way junction;
said second end of said third capillary tube being connected to a fraction collector by a first port of a “T” fitting;
a nozzle fluidly attached to a second port of said “T” fitting, and said nozzle being connected to an electrical ground by a grounding wire;
said fraction collector being supplied with a sheath liquid; said sheath liquid being filtered by an inline filter fluidly connected to a sheath buffer reservoir;
said inline filter being fluidly connected to a computer-controlled valve that is itself fluidly connected to a third port of said “T” fitting;
a collection vessel on a translational stage which moves in at least one of the X, Y, and Z directions disposed to receive fluid dispensed from said nozzle.

10. The apparatus for identifying a polynucleotide capable of binding a target of claim 9, wherein said first buffer channel and said first waste outlet are perpendicular to said first capillary tube and said second capillary tube.

11. The apparatus for identifying a polynucleotide capable of binding a target of claim 9, wherein said second buffer channel and said second waste outlet are perpendicular to said second capillary tube and said third capillary tube.

12. The apparatus for identifying a polynucleotide capable of binding a target of claim 9, wherein said second buffer channel and said second waste outlet are parallel to said first buffer channel and said first waste outlet.

13. The apparatus for identifying a polynucleotide capable of binding a target of claim 9, further comprising a fraction collector fluidly connected to a second end of said third capillary tube.

14. The apparatus for identifying a polynucleotide capable of binding a target of claim 11, further comprising a computer program capable of controlling said first high voltage power supply, said second high voltage power supply, said third high voltage power supply, said injector, and said fraction collector.

15. The apparatus for identifying a polynucleotide capable of binding a target of claim 9, wherein the collection vessel is a 96-well plate.

16. The apparatus for identifying a polynucleotide capable of binding a target of claim 9, wherein the first four-way junction is sealed.

17. The apparatus for identifying a polynucleotide capable of binding a target of claim 9, wherein the second four-way junction is sealed.

18. The apparatus for identifying a polynucleotide capable of binding a target of claim 9, wherein the sheath buffer reservoir is held under pressure greater than atmospheric pressure.

19. The apparatus for identifying a polynucleotide capable of binding a target of claim 18, wherein the pressure greater than atmospheric pressure is provided by pressurized nitrogen gas.


SEQUENCE LISTING (8 sequences)

SEQ ID NO: 1 - DNA, 15 bp, Artificial Sequence
  thrombin binding aptamer
ggttggtgtggttgg

SEQ ID NO: 2 - DNA, 20 bp, Artificial Sequence
  primer number 1 for pool of polynucleotides
agcagcacagaggtcagatg

SEQ ID NO: 3 - DNA, 20 bp, Artificial Sequence
  primer number 2 for pool of polynucleotides
cctatgcgtgctaccgtgaa

SEQ ID NO: 4 - DNA, 20 bp, Artificial Sequence
  primer number 3 for pool of polynucleotides
agcagcacagaggtcagatg

SEQ ID NO: 5 - DNA, 20 bp, Artificial Sequence
  primer number 4 for pool of polynucleotides
ttcacggtagcacgcatagg

SEQ ID NO: 6 - DNA, 58 bp, Artificial Sequence
  Illumina′s TruSeq Universal Adapter
aatgatacggcgaccaccgagatctacactctttccctacacgacgctcttccgatct

SEQ ID NO: 7 - DNA, 63 bp, Artificial Sequence
  TruSeq Adapter, Index 6
gatcggaagagcacacgtctgaactccagtcacgccaatatctcgtatgccgtcttctgcttg

SEQ ID NO: 8 - DNA, 29 bp, Artificial Sequence
  thrombin binding aptamer
agtccgtggtagggcaggttggggtgact